(19) United States
(12) Patent Application Publication
(10) Pub. No.: US 2019/0231217 A1
(43) Pub. Date: Aug. 1, 2019

(54) DEVICE FOR THE EXAMINATION OF LIMB JOINTS

(71) Applicant: IDDMEDICAL SP. Z O.O., Bielsko-Biala (PL)

(72) Inventors: Boguslaw SADLIK, Bielsko-Biala (PL); Anna PODLESNY, Bielsko-Biala (PL); Andzej PODLESNY, Bielsko-Biala (PL)

(73) Assignee: IDDMEDICAL SP. Z O.O., Bielsko-Biala (PL)

(21) Appl. No.: 16/318,129

(22) PCT Filed: Jul. 14, 2017
(86) PCT No.: PCT/PL2017/000072

§ 371 Date: Jan. 15, 2019

(30) Foreign Application Priority Data
Jul. 15, 2016 (PL) P.417 984

(51) Int. Cl. A61B 5/055; A61B 6/03; A61B 6/04; A61B 5/00

(52) U.S. Cl. CPC A61B 5/0555; A61B 5/4528; A61B 6/04; A61B 6/032

(57) ABSTRACT

A device for examining limb joints comprises a support frame to which are attached mechanisms with holders for positioning patient's limbs in the region encompassing patient's hips, thighs and feet, said mechanisms being made of materials that do not interact with magnetic fields. A hip belt (B) is secured to special ports on both sides of the support frame (A) of the device, then a thigh support platform (C) is secured to the support frame on a swingable hinge, and a shank support assembly (D) is secured in the middle part, where on a common adjustment screw guided by a slider and locked in position on the support frame by means of a position nut is located a fixed casing combined with a nut and with two force measurement assemblies. A feet support assembly (E) is secured to the opposite side of the frame. The second variant of the device consists of only the following groups: the support frame (A), the thigh support platform (C) and the shank support assembly (D).

The invention relates to a device for examining the lower limbs of patients, such device being designed to be used with apparatus for magnetic resonance and computed tomography examinations.

Nowadays, magnetic resonance scanners enable very accurate imaging of extremely diverse tissues and anatomical structures of patients, while retaining the non-invasive nature of such procedures. In relation to previously used methods, such as two-dimensional examination using ionising radiation (X-ray), this improvement has a number of advantages, as listed below:
it enables obtaining very detailed, high definition photographs of virtually any tissue in the human body,
it enables obtaining photographs of high contrast, also of soft tissues,
it is safe for almost every patient in every state, the exception being people with artificial cardiac pacemakers and/or with foreign structures having magnetic properties,
examinations may be performed at short intervals because of the low risk of undesirable effects resulting from the examination,
the results of the examination are images of sections in various planes obtained without changing the position of the patient.

These and many other advantages make modern imaging techniques (including magnetic resonance imaging (MRI) and computed tomography (CT)) increasingly important. They are applied in the diagnostics of an increasing number of diseases.

The growing popularity of imaging diagnostics is reflected in the rapidly expanding market of MRI apparatus and additional equipment.

Despite the many advantages of high definition imaging, it often happens, in medical practice, that images of bone/skeletal structures, or of soft tissues under static examination, are blurred, making it difficult or impossible to correctly diagnose. Usually, the underlying causes include excessively dense arrangement of structures that conceal the areas of potential damage and the position of the mating elements remaining at rest in close proximity to each other. The visibility of soft tissue damage improves greatly if specific relative positions of the mating articular structures are attained through the action of external force. The result of examination is even easier to interpret when both limbs are put together side by side. If both limbs are fixed and subjected to load in an identical manner, comparative observation of symmetrical structures is possible. The load increases the distance between the anatomical structures, and the difference with respect to the undamaged joint precisely determines the degree of injury.

Medical diagnostic devices performing very similar functions are known, but their characteristics compared to the presented device are different.

Usually devices of similar range of capabilities are the subject of patents pertaining to examinations with the use of X-rays, where two-dimensional images (traditional X-ray examination) are obtained. Other devices are designed to handle a single joint or one limb only. The following are a few patents that in part of their scope include descriptions of solutions similar to some extent to that of the present invention.

U.S. Pat. No. 4,232,681 describes a device for positioning a leg for X-ray examinations, where only one position of the examined leg is possible and only one force can be exerted on the leg. The device comprises a fixing platform with a holder for supporting the lower portion of the limb, and a second holder, in fact a side wall, for fixing the knee from one side. On the side, between the two holders, there is an extendable element which applies the required force to the limb between the holders.

Similar solutions are disclosed in patents:

U.S. Pat. No. 4,291,229—diagnosis of meniscus of one leg in a forced position,

U.S. Pat. No. 5,445,152—MRI examination of one knee in several positions,

U.S. Pat. No. 6,499,484—MRI examination of damaged ligaments of the knee of one leg,

U.S. Pat. No. 7,036,169—MRI examination, both lower extremities at the same time, one strictly defined position,

DE102011007041—positioning of an additional coil of an MRI apparatus for the knee with variable angle of flexion.

None of the solutions presented above provides all the capabilities that are necessary to resolve problems such as:
ability to carry out comparative examinations of both lower limbs at the same time, under same load,
ability to exert loads of accurate known value on mating structures of the lower limbs. Loads can be adjusted within a defined range,
positions can be adjusted during the examination for every structure of both lower limbs.

In the field of examining the functional fitness and damage to the human lower limbs, the answer to physician's observations is the device described below, which is the subject-matter of this patent.

The essence of the invention is a device for examining limb joints, said device including a support frame to which are attached mechanisms with holders for positioning patient's limbs in the region encompassing patient's hips, thighs and feet, said mechanisms being made of materials that do not interact with magnetic fields. A hip belt is secured to special ports on both sides of the support frame of the device, then a thigh support platform is secured to the support frame on a swingable hinge, and a shank support assembly is secured in the middle part, where on a common adjustment screw guided by a slider and locked in position on the support frame by means of a position nut is located a fixed casing combined with a nut and with two force measurement assemblies, while a feet support assembly is secured to the opposite side of the frame. The hip belt has two side straps provided with carabiners attached to ports on both sides of the support frame. Supports for adjustable pads are secured to the shank support assemblies, and adjustable support pads are mounted slidably in the vertical direction on the supports for adjustable pads. The relative vertical positions of the adjustable support pads are adjusted by means of load adjustment knobs. The vertical position of the entire casing combined with a nut and with two force measurement assemblies and of all elements mounted on the casing is adjusted by means of a turning handle. The thigh support platform has two lower hinges mounted onto the support frame for allowing the thigh support platform to tilt. The thigh support frame is secured in a selected angular position by means of angular position locks located near each of the lower hinges. The thigh pad platform moves along guides combined with the rotating part of the lower hinge. The position of the thigh pad platform is set by means of thigh position locks located on both sides of the platform. On the thigh pad platform there are two thigh pads which can be moved towards the sides of the device, and they are locked in position by means of thigh pad locks of each pad. Each thigh pad has its strap with a protective pad. The feet support assembly has two guide fastening blocks secured to the support frame. A feet spacing guide arranged along the device is secured in guide fastening blocks. The remaining elements of the feet support assembly can be moved along the feet spacing guide. These elements can be locked in an initial position in the longitudinal direction by means of an initial position latch. The position is adjusted finely by a feet load adjustment screw operated by an adjustment crank, and the value of the patient's feet support reaction force is determined on a feet reaction force measurement support. On the two feet spacing guides and on the spacing adjustment screw there are two foot holders and foot holding straps with protective pads attached to the foot holders.

The distance between the foot holders can be adjusted by means of a feet spacing adjustment crank. Each of the foot holders is rotatable around its rotation axis, and it is locked in an angular position by means of a foot torsion angle position latch. Both foot holders mounted on the feet spacing guides are rotatable in relation to the lower guide. The angle of rotation can be set and locked by means of a middle connector with an angular position lock. The relative spacing between foot holders is adjusted by the spacing adjustment screw.

The device disclosed herein extends the diagnostic capabilities of MRI apparatus and tomographs by improving the quality of images obtained and their clarity for orthopaedists and radiologists. The device enables imaging of damage that is usually invisible or inconclusive in a standard examination. This effect is achieved by precisely constraining the position of specific parts of the lower limbs so as to produce an intended stress of known magnitude in the examined tissue system. It must be borne in mind that the magnitude and range of forces that create these loads are proportional to the effects obtained. At the same time these forces must be adapted to the patient's weight and size. For instance, the loads applied in the examination of children and adolescents must be correspondingly lower than those applied in the examination of adult men. The device is provided with means for the evaluation of the loads the patient is subjected to.

Similarly, the relative positions of the elements that determine the posture of the examined person must be adapted to the anthropometric parameters of the particular patient. For this reason each of the supports is provided with means for adjusting its position and size so as to enable optimum adaptation to the height and bulkiness of the examined patient.

A specific feature of the device is its capability of positioning and securing of patient's both lower limbs in any required position. The position of the lower limbs is defined by the following parameters:
angle between the patient's torso and the patient's thighs,
point of support and restraining of thighs,
angle between the thighs,
angle between thigh and shank (knee joint),
angle between shank and foot (ankle),
spacing between feet,
angle of foot torsion relative to the shank (ankle).

It is possible to precisely specify the value of each of these parameters. The range of possible values is determined by the physiological abilities of a particular patient.

The design solutions applied in the device, wherein the positions of both limbs are identical, enables comparative examination of the limbs.

The forced position allows for accurate imaging of damages of intra-articular and extra-articular structures that are not visible or are poorly visible in static no-load examination. Joint immobilisation also allows for the elimination of the patient's motion artifacts, significantly improving the sharpness of the image, and thereby the quality of the examination.

Other advantages and features of the device:
ability to adapt to virtually any MRI apparatus or computer tomograph with minimum structural modifications (additional adapter package comprising latches and positioning pins),
wide adaptability and versatility of use, the design enables diagnosing patients of various body structure and height,
wide range of diagnosable injuries—as the lower limbs are positioned from the hips level down, this enables MRI or CT scanning of virtually every element and joint within this area,
ability to conduct comparative examinations—symmetrical alignment of the left and the right lower limb allows for diagnosis by comparing the damaged structure with the healthy one,
no interaction with the MRI apparatus and no shadowing of the examined area, due to the materials used to construct the platform,
easy assembly and dismantling,
intuitive adjustment and fixing of the patient—standardised procedures and elements applied in the device:
methods of positioning the individual sections—latch pins,
fixing of the patient—straps fastened on the limb,
fine adjustments—screws with marking and force indicators.

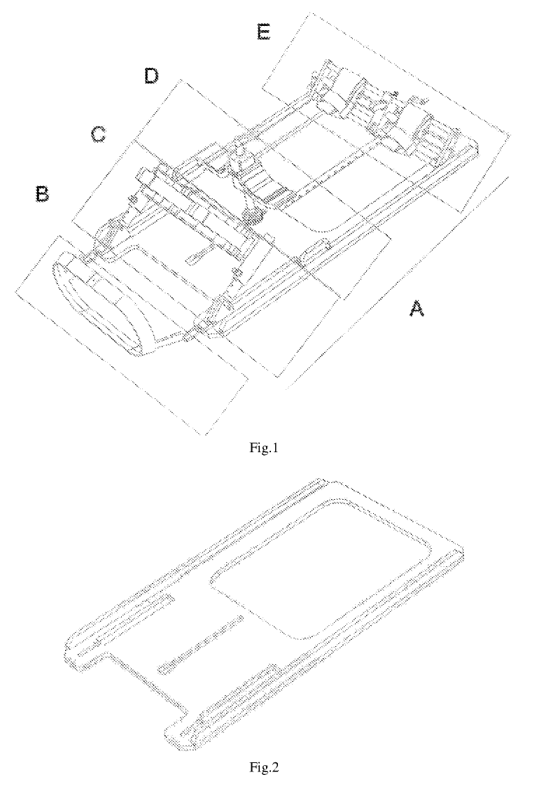

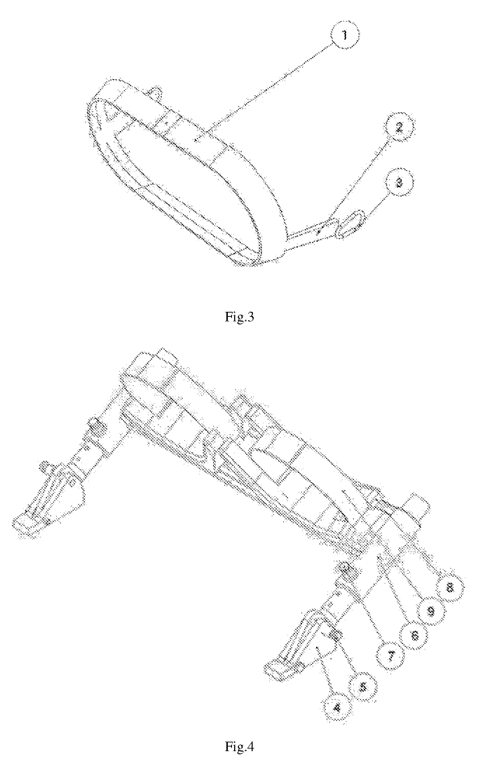

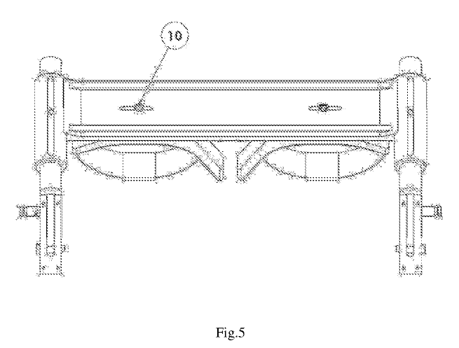

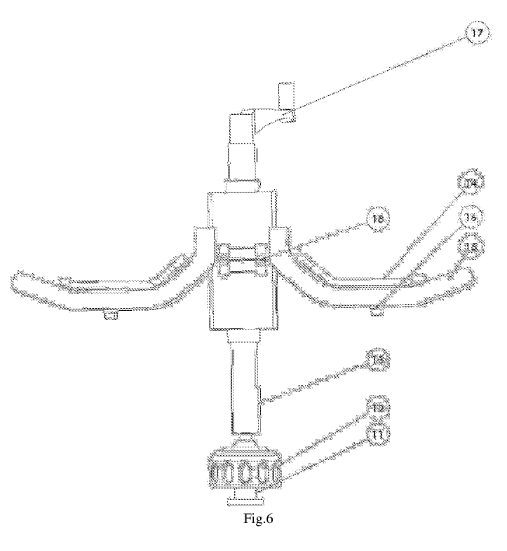

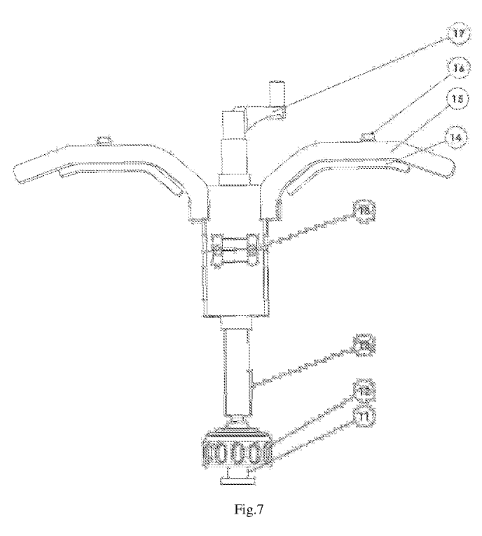

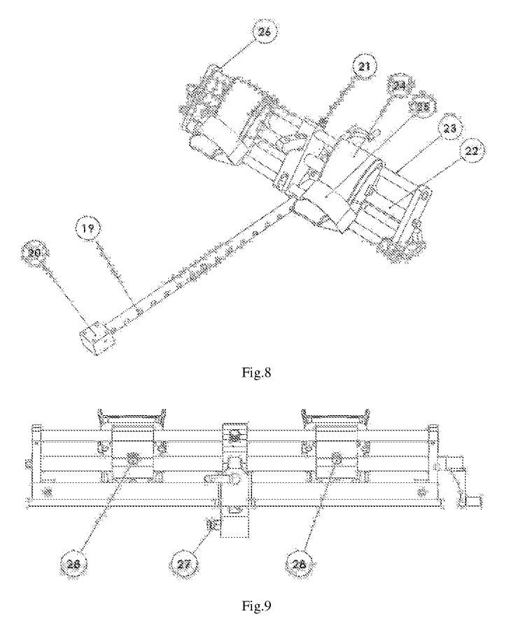

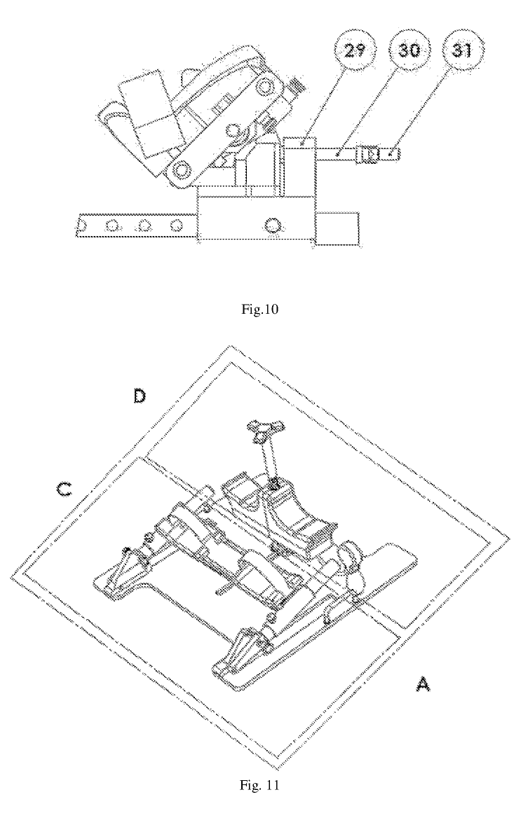

The subject of the invention is illustrated by an embodiment shown in drawings, of which FIG. 1 depicts an axonometric view of the device and illustrates a breakdown into functional groups, FIG. 2 depicts the support frame, FIG. 3 depicts the hip belt, FIG. 4 depicts the thigh support platform, FIG. 5 is a rear view of the thigh support platform, FIG. 6 depicts the shank support assembly where the acting force is oriented upwards, FIG. 7 depicts the shank support assembly where the acting force is oriented downwards, FIG. 8 depicts the feet support assembly with the positioning mechanism, FIG. 9 is a rear view of the feet support assembly with the positioning mechanism, FIG. 10 is a side view of the feet support assembly with the positioning mechanism.

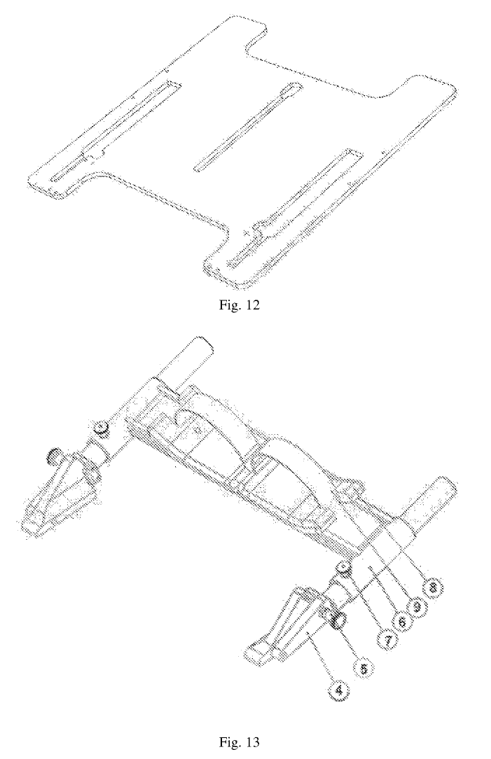

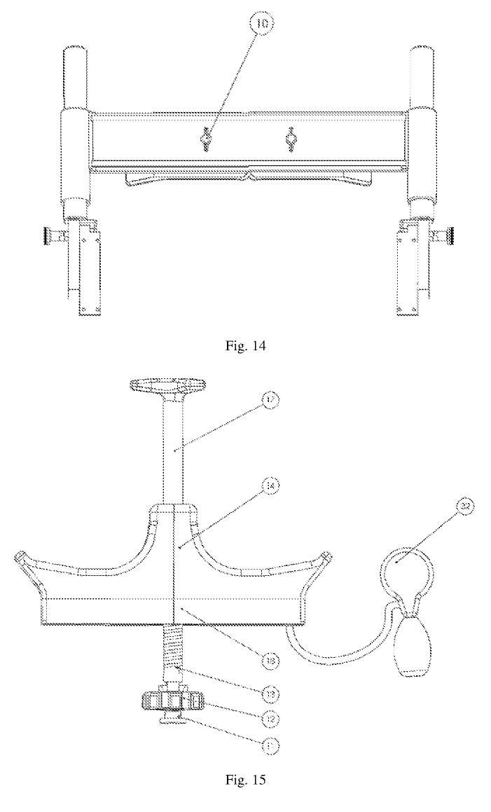

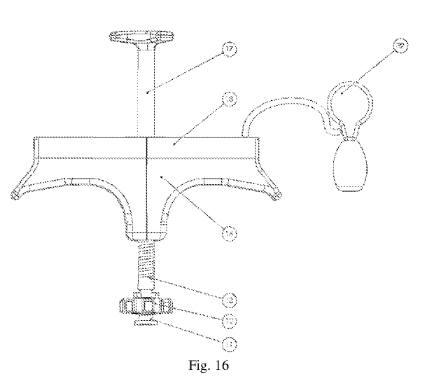

A second variant of the invention is illustrated by an embodiment, where FIG. 11 depicts an axonometric view of the device and illustrates a breakdown into functional groups, FIG. 12 depicts the support frame of reduced size, FIG. 13 depicts the thigh support platform, FIG. 14 is a rear view of the thigh support platform, FIG. 15 depicts the shank support assembly where the acting force is oriented upwards, FIG. 16 depicts the shank support assembly where the acting force is oriented downwards.

The device comprises groups of devices and mechanisms set up as follows:

A—support frame,

B—hip belt with support frame fastener,

C—thigh support platform,

D—shank support assembly,

E—feet support assembly with positioning mechanism.

In its second variant the device may consist of only the following groups: A, C and D, without hip belt with support frame fastener (B) and feet support assembly with positioning mechanism (E) (FIG. 11).

Every group performs specific functions, and the functionality of the entire device is the effect of the operation of its individual components.

The structure, components and functions of each group are described below.

Support frame A—basic component of the device. It is placed directly on the bed of the examination apparatus. Special shape elements on the underside of the frame serve for transverse positioning of the frame in the grooves of the bench of the examination apparatus. The support frame is movable along the bed of the examination apparatus to match its position to the patient's position. All the remaining sections of the device are attached to the support frame. It acts as an element which transfers loads between the sections of the device, it is entirely made of materials that do not interact with magnetic fields and at the same time are rigid and lightweight.

In the second variant of the device the support frame has a reduced size FIG. 12.

The hip belt B confines the movements of platform C along the guide grooves in the bed of the examination apparatus.

Forcing the lower limbs to adopt a specific position causes them to act on the support frame A with a force that could move the frame away from the patient. The hip harness counteracts such movement.

This section consists of:
1. Hip belt proper
2. Two side straps
3. Three carabiner latches

The installation of the hip belt consists in adapting its length to the patient's dimensions and mounting it on the patient's hips. The belt 1 can be mounted either when the patient is standing prior to positioning the patient on the bench of the apparatus, or when the patient is lying. The patient is then positioned on the bed of the MRI apparatus and the hip belt is fastened with side straps 2 by means of special carabiners 3 locked onto dedicated ports on both sides of platform C.

The lengths of side straps 2 can still be adjusted at this stage to take up possible slack. This facilitates positioning of the limbs on the platform at a later stage.

When diagnostic tasks are completed, it just suffices to unfasten the hip belt 1 to free the patient.

The thigh support platform C is the first of the sections designed to precisely position the patient's lower limbs.

This section consists of:
4. Lower hinge
5. Angular position lock
6. Thigh pad platform
7. Thigh platform position lock
8. Thigh pad
9. Thigh strap with protective pad
10. Thigh pad position lock

The fixed parts of the two lower hinges 4 are secured to the support frame of the device. The moving parts of the mechanism enabling adjustment of the angular position of the thigh support are arranged coaxially in these components. The angle between the base of the device and the thighs is fixed by locking the angular position lock 5 in one of the several available positions. This angle can be changed stepwise from 0 to ca. 45°. The set angle depends on the purpose of the examination. The thigh pad platform 6 moves along guides of the moving part of the lower hinge 4. Upon setting the platform in the desired position it must be secured in that position by means of thigh platform position locks 7 on both sides of the device. The thigh pads 8 are designed to support the patient's thighs and arrange them in a selected position. The spacing between the pads on the platform can be adjusted by moving the pads closer to or away from each other to adapt that spacing to patient's anthropological features, patient's dimensions and to the purpose of the examination. Upon setting the desired position, the pads can be secured by means of thigh pad position lock 10, separately for the left and the right pad. Locks 10 are located at the back of the thigh pad platform 6. Upon setting all parameters of the position of patient's limbs within the thigh section, the thighs can be secured. This is done by fastening the patient's thighs to the previously arranged section by means of thigh straps 8 which are attached to the thigh pads 9. The fastening is effected by tightening the thigh straps 9 with protective pads to restrict the free movement of patient's limbs during the setting of subsequent sections of the device and throughout the examination.

Upon completing the examination the thigh straps 9 must be unfastened before the patient can leave the bench of the device. When the angle between the thighs and the base of the device is set to higher values, the structural components of the thigh supporting section may make leaving the bench by the patient difficult. In such case it is advisable to release both angular position locks 10 so that the thigh section falls to neutral position.

The shank support assembly D may take on one of two possible configurations, depending on the purpose of the examination. This section has two independent shank supports mounted on one common adjustment screw.

The shank support assembly consists of:
11. Guide slider
12. Position nut
13. Adjustment screw
14. Support pad, adjustable—symmetrically for both limbs
15. Adjustable pad support—symmetrically for both limbs
16. Load adjustment knob—symmetrically for both limbs
17. Central adjustment turning handle (crank)
18. Casing combined with a nut and with two force measurement assemblies—symmetrically for both limbs

In the second version of the device the shank support assembly consists of:
11. Guide slider
12. Position nut
13. Adjustment screw
14. Support pad, adjustable—symmetrically for both limbs
17. Central adjustment turning handle (crank)
18. Casing combined with a nut and with two force measuring assemblies—symmetrically for both limbs
32. Indicator of the total load applied to both limbs

The shank support assembly may be installed on the platform at any time. It is installed by inserting the guide slider 11 into a special socket at the end of a special guide on the support frame of the platform. It is desirable that the force applied by the assembly to the limb acts along the intended direction, which is coincident with the axis of the shank support assembly. When comparative examinations of the structures of both limbs are conducted, both shanks should be supported in a similar manner.

Upon installing the shank support assembly D onto the support frame A and upon selecting its position on the guide, the assembly should be secured in position by tightening the position nut 12. The installation position of the assembly can be readjusted at any stage of using the device by loosening the position nut 12, repositioning the assembly and retightening the nut.

This should be followed by mounting the components that will come into contact with the limbs. The casing combined with a nut and with two force measuring assemblies 18 is mounted onto the adjustment screw 13. The force measuring assemblies enable measurement of the force applied to the support pads 14, separately for each limb.

Then, by turning the crank 17, the shank support pads 15 should be moved into an initial position matching the position of the patient's leg. The force measurement assemblies 18 will not always indicate identical values. The device allows the limb support to be adjusted so that loads on both legs are identical. Turning the load adjustment knob 16 clockwise moves the adjustable pad 14 away from the adjustable pad support 15 increasing thereby the force applied to the limb, the value of that force being determinable by means of the force measurement assembly 18.

In the second variant of the device the force applied is measured jointly for both limbs by means of indicator 32, whereas the design of the combined casing 18 causes symmetrical distribution of the load between both limbs. Therefore the force applied to one limb constitutes half of the value displayed by indicator 32.

In the second version of the device the desired load on both limbs is applied by turning the crank 17 while observing the indicator 32.

Applying a force of defined value and direction of action to a limb improves the visibility of potential damages in that limb's locomotor system. The device described herein, as a whole, enables to fulfil such functions. In the case of comparative examinations between two limbs, it is important that the manner of applying force, its direction and value are as close as possible for both limbs.

The last section E, the feet support assembly, is designed to lock patient's feet in a defined position.

That section comprises:
19. Longitudinal position guide
20. Guide fastening block
21. Feet spacing guide
22. Spacing adjustment screw
23. Middle connector with an angular position lock
24. Foot holder—symmetrically for both limbs
25. Foot fastening strap with protective pad—symmetrically for both limbs
26. Feet spacing adjustment crank
27. Initial position lock
28. Foot torsion angle position latch
29. Feet reaction force measurement support
30. Feet load adjustment screw
31. Adjustment crank

The positioning of the assembly starts with the setting of its longitudinal position. The optimal position should be adapted to the features of the patient while moving the entire section along the longitudinal position guide 19. The longitudinal position guide 19 is secured in two guide fastening blocks 20 on both ends of the guide, said blocks constituting at the same time position limiters for the entire assembly. The next entire section is locked by means of initial position lock 27. Depending on the settings of the previous sections, the angle between the base plane and the feet supporting surfaces should be determined. The device enables setting this angle stepwise from 90° to 45°. Such settings correspond to natural arrangement of feet ranging from the position in which the legs are straightened to the angle of 90° in the knee. The angle position is secured by means of the middle connector with an angular position lock 23. The feet section enables setting the spacing between feet to adapt the arrangement of patient's limbs on the platform to the type of examination. The required distance between patient's feet is adjusted by means of the removable feet spacing adjustment crank 26. Clockwise turning of the crank symmetrically increases the spacing, anticlockwise turning decreases the spacing.

The device also enables setting the torsion angle of the entire foot on the platform. That angle can be adjusted separately for each foot. Releasing the foot torsion angle position latch 28 enables turning the foot holder 24 by up to 45° in both outward and inward directions. The angle is adjusted in a stepwise manner and upon setting the desired position the foot holder 24 is locked in that position by releasing the foot torsion angle position latch again.

In order to prevent changes in the position of patient's feet during load adjustment and during the examination, the fastening straps with protective pad 25 should be fastened.

If an initial load is to be applied during the examination, the load is applied to the limbs in the longitudinal direction up to the required value by means of the adjustment crank 31 operating the feet load adjustment screw 30. The combined feet reaction force measurement support 29 enables determining the value of the force applied.

1. A device for examining limb joints, said device including a support frame to which are attached mechanisms with holders for positioning patient's limbs in the region encompassing patient's hips, thighs and feet, said mechanisms being made of materials that do not interact with magnetic fields, characterized in that a first variant of the device consists of: hip belt (B) which is secured to special ports on both sides of the support frame (A) of the device, then a thigh support platform (C) is secured to the support frame on a swingable hinge, and a shank support assembly (D) is secured in the middle part, where on a common adjustment screw (13) guided by a slider and locked in position on the support frame (1) by means of a position nut (12) is located a fixed casing combined with a nut and with two force measuring assemblies (18), while a feet support assembly (E) is secured to the opposite side of the frame, as well as second variant of the device consists of: the support frame (A) of the device, then a thigh support platform (C) is secured to the support frame on a swingable hinge, and a shank supped assembly (D) is secured in the middle part, where on a common adjustment screw (13) guided by a slider and locked in position on the support frame (1) by means of a position nut (12) is located a fixed casing combined with a nut and with two force measuring assemblies (18).

2. The device according to claim 1 characterized in that the hip belt (B) has two side straps (2) provided with carabiners (3) attached to ports on both sides of the support frame (A), or that the hip belt is not present in the second variant of the device, which comprises groups of devices and mechanisms set up as follows:
A—support frame (in reduced size),
C—thigh support platform,
D—shank support assembly.

3. The device according to claim 1 characterized in that supports for adjustable pads (15) are secured to the shank support assemblies (D), and adjustable support pads (14) are mounted slidably in the vertical direction on the supports for adjustable pads, the adjustable support pads being adjustable by means of a load adjustment knob (16).

4. The device according to claim 1 characterized in that the fixed casing combined with a nut and with two force measurement assemblies (18) is adjusted in the vertical plane by means of knob (17).

5. The device according to claim 1 characterized in that the thigh support frame (C) is secured rotatably to two lower hinges (4) attached to the support frame (A), while the selected angular position is set by means of angular position locks (5) located near each of the lower hinges (4).

6. The device according to claim 5 characterized in that a thigh pad platform (6) is set on guides combined with the rotating part of the lower hinge (4) and the position of the thigh pad platform (6) is set by means of thigh position locks (7) located on both sides of the platform.

7. The device according to claim 5 characterized in that on the thigh pad platform (6) there are two thigh pads (8) which can be moved towards the sides of the device, and they are locked in position by means of thigh pad locks (10) of each pad.

8. The device according to claim 7 characterized in that each thigh pad (8) has its thigh strap with a protective pad (9).

9. The device according to claim 1 characterized in that the feet support assembly (E) has two blocks (20) for fastening a longitudinal position guide (19) secured to the support frame (A), the said longitudinal position guide having installed on it a feet spacing guide (21) with a spacing adjustment screw (22), a middle connector with an angular position lock (23), a foot holder (24)—symmetrically for both limbs, and a feet spacing adjustment crank (26), which can be locked in an initial position in the longitudinal direction by means of an initial position latch (27), or that the feet support assembly is not present in the second variant of the device, which comprises groups of devices and mechanisms set up as follows:
A—support frame (in reduced size),
C—thigh support platform,
D—shank support assembly.

10. The device according to claim 9 characterized in that the precise position of the feet spacing guide (21) is set by means of a feet load adjustment screw (30) with an adjustment crank (31).

11. The device according to claim 9 characterized in that each foot holder (24) is mounted rotatably in relation to its rotation axis, and its angular position is set by a foot torsion angle position latch (28), whereas the rotation of the foot holder (24) on the feet spacing guides (21) is set by means of the middle connector with an angular position lock (23), and whereas the relative spacing of foot holders (24) is set by the spacing adjustment screw (22).

12. The device according to claim 4 characterized in that it enables measurement of the applied force by means of indicators on the casing (18) or by means of an indicator of the total load applied to the limbs (32).
